(19) United States
(12) Patent Application Publication
(10) Pub. No.: US 2023/0383251 A1
(43) Pub. Date: Nov. 30, 2023

(54) METHODS FOR THE NANOCONFINED CULTIVATION OF T-, B- AND NK- CELLS

(71) Applicant: ETH ZURICH, Zurich (CH)

(72) Inventors: Morteza ARAMESH, Dubendorf (CH); Diana STOYCHEVA, Zurich (CH); Enrico KLOTZSCH, Zurich (CH)

(21) Appl. No.: 18/249,013

(22) PCT Filed: Oct. 11, 2021
(86) PCT No.: PCT/EP2021/078005

§ 371 Date: Apr. 13, 2023

(30) Foreign Application Priority Data
Oct. 15, 2020 (EP) 20201972.5

(51) Int. Cl. C12N 5/0783; C12N 5/0781

(52) U.S. Cl. CPC C12N 5/0636; C12N 5/0635; C12N 5/0646; C12N 2535/00; C12N 2533/50; C12N 2533/10

(57) ABSTRACT

The present invention is directed to a method for the cultivation, optionally activation and growth of lymphocytes (T-, B- and NK-cells) by culturing these cells in a suitable cell growth medium on a nanoporous substrate having a pore diameter in the range of about 100 to 500 nm, optionally about 150 to 250 nm.

The present invention is directed to a method for the cultivation, optionally activation and growth of lymphocytes (T-, B- and NK-cells) by culturing these cells in a suitable cell growth medium on a nanoporous substrate having a pore diameter in the range of about 100 to 500 nm, optionally about 150 to 250 nm.

Many cells can be cultured and grown in culture media while adhering to solid surfaces. Nanoporous surfaces increase the surface area and can be tailored with a wide range of surface modifications. For example, nanoporous anodic aluminum oxide (AAO) nanopores can be formed from inexpensive anodization of pure aluminum which results in the self-assembly of highly ordered, vertical nanochannels with well-controllable pore diameters, depths and interpore distances. Nanoporous substrates such as AAO substrates, also called nanoporous AAO membranes, have gained utility as cell interfaces in many bioapplications, for example, as biofiltration membranes, lipid bilayer support, implant coatings, drug delivery systems with AAO capsules and scaffolds for tissue engineering. Nanoporous membranes have low production costs and are commercially available with pore diameters of 20 to 400 nm. For example, the article of D. Brueggemann in the J. of Nanomaterials, Vol. 2013, Art. ID 460870, 1 to 18, reviews previous studies on the AAO support-based cell growth of a number of cell and cell tissue types including neuronal cells, connective tissue cells, epithelial cells, muscle cells and blood cells as well as microorganisms. Nanotopographic features of a biomaterial such as AAO influence its interaction with biological tissues or cells for cell culture applications. The coating and pore diameters of AAO materials as well as the intention for culturing cells therewith varies widely with the cells and their intended applications. For example, 200 nm pore diameters support neurite outgrowth and 400 nm pore diameters actually accelerate axon growth, osteoblasts grow extensions into 75 and 89 nm pore diameter AAOs, epithelial cells proliferate and adhere best on AAO with pore diameters up to 45 nm, whereas muscle cell proliferation proved better on 200 nm than on 20 nm pores. For blood cells the optimal pore diameter for activation and proliferation varied with the cell type from 20 to 400 nm. In summary, nanoporous substrates are known to alter many biological processes of very different cells including proliferation, growth, adhesion, differentiation, secretion and migration (see D. Brueggemann above).

Cai et al., Nature Nanotechnology, vol. 13, July 2018, 610 to 617 teaches the use of nanofabricated single-molecule array platforms in two different configurations, (a) with monovalent TCR ligands arranged co-planar with a supported lipid bilayer featuring mobile adhesion molecules (2D), thus excluding the CD45 transmembrane tyrosine phosphatase, or (b) with monovalent TCR ligands elevated by 10 nm on solid nanopedestals (3D), allowing closer access of CD45 to engaged TCR in order to control ligand geometry both in-plane and out-of-plane with these two planar and elevated arrangements of monovalent TCR ligands in order to study the role of ligand geometry in TCR triggering and to identify the contributions of lateral and axial components of ligand positioning.

Lymphocytes are a type of white blood cell in the vertebrate immune system and include natural killer (NK)-cells (for cell-mediated, cytotoxic innate immunity), T-cells (for cell-mediated, cytotoxic adaptive immunity), and B cells (for humoral, antibody-driven adaptive immunity). These three major types of lymphocytes differentiate from a common lymphoid progenitor cell.

B-cells, also termed B lymphocytes are lymphocytes that secrete antibodies and define the humoral immunity of the adaptive immune system. Additionally, B-cells present antigens and secrete cytokines. B-cells express B cell receptors (BCRs) on their cell membrane. BCRs allow the B-cell to bind to a specific antigen, against which it will initiate an antibody response. B-cells are an important source of commercially relevant pharmaceutical antibodies for treating, preventing and identifying diseases. B-cells can be genetically engineered to produce an antibody with a desired specificity, such as hybridoma B-cells.

T-cells are a type of lymphocyte that develops in the thymus and has a central role in the adaptive immune response. T-cells present a T-cell receptor (TCR) on their cell surface that recognizes specific epitope of a foreign antigen. T-cells migrate as precursor cells from the bone marrow to the thymus and develop into either CD8+ cytotoxic T-cells or CD4+ helper T-cells that convey a variety of immune-related functions. For example, CD8+ cytotoxic T-cells cause immune-mediated cell death in e.g. virus-infected cells or cancer cells. CD8+ T-cells also secrete cytokines to recruit other immune-related cells. CD4+ T-cells function as “helper cells” by secreting cytokines or providing receptor-mediated signaling to navigate the immune response. They play a crucial role in initiating and controlling most B-cell responses. Regulatory or also called suppressor CD4+ T-cells are yet another distinct population and convey self-tolerance by distinguishing invading cells from “self”, thus preventing an autoimmune response.

T-cells are the basis of adoptive immunotherapy, a therapeutic approach, wherein T-cells are isolated, modified, activated and re-implanted into a patient to selectively kill specific pathogenic target cells, such as cancer or infected cells. For example, T-cells genetically engineered to express chimeric antigen receptors (CARs) have proven an impressive therapeutic activity in patients with certain cancer types (see Rafiq et al., Nature Reviews/Clinical Oncology, vol. 17, March 2020, 147-167). T-cell activation is a complex process, involving both biochemical as well as mechanical cues that define the T-cell response and eventually treatment effectiveness.

Natural killer (NK)-cells, also known as large granular lymphocytes (LGL), are part of the innate immune system. Similar to cytotoxic T cells in the adaptive immune response, NK cells rapidly detect and kill infected or altered cells, which makes them a very attractive tool for clinical applications. Unlike T-cells, NK cells do not express antigen-specific receptors but utilize inhibitory receptors (killer immune-globulin-like receptors and Ly49) to develop and to recognize infected or altered cells. In this way they provide a broad and immediate line of protection. NK cells can be identified by the presence of CD56 and the absence of CD3 (CD56⁺, CD3⁻).

T-, B- and NK-cell activation and expansion is presently performed in solution on flat surfaces or in solution via microbeads or dendrimers for providing activation-specific antibodies.

In view of the above, the objective of the present invention is to provide further cell types that are suitable for nanopore substrate-based cultivation, in particular suitable for the cost-effective nanopore-based cultivation, and/or to provide an improved cultivation method for lymphocytes, i.e. NK-, T- and/or B-cells. It is a further objective to improve lymphocyte activation and antibody production.

In a first aspect, this objective is solved by a method for the cultivation, optionally activation and growth, of lymphocytes (T-, B- and NK-cells), i.e. for the cultivation of T-, B- and/or NK-cells, comprising the step of culturing the cells in a suitable cell growth medium on a nanoporous substrate, characterized in that the nanoporous substrate has a pore size, i.e. pore diameter, in the range of about 100 to 500 nm, optionally about 150 to 400 nm or 150 to 250 nm, optionally of about 200 nm.

The term B-cells in the present invention is used as commonly accepted and encompasses all B-cell subtypes, such as naive, activated, germinal-center, regulatory, plasma as well as memory B-cells. The term further encompasses engineered B-cells, including hybridoma, optionally for use in antibody production.

The term T-cells for use in the present invention is used as commonly accepted and encompasses all T-cell subtypes: CD8+ T-cells (“killer cells”), CD4+ T-cells (“helper cells”) as well as regulatory T-cells (“suppressor T-cells), all of which express a T-cell receptor on their cell surface. The term further encompasses engineered T-cells including CAR T-cells, optionally for use in CAR T-cell therapy.

The term NK-cells for use in the present invention is used as commonly accepted and encompasses all NK-cell splice variants, all of which are characterized by the presence of CD56 and the absence of CD3 (CD56⁺, CD3⁻). The term further encompasses engineered NK-cells including CAR-NK cells, optionally for use in cancer NK-cell therapy.

The term “culturing cells in a suitable cell growth medium is used as commonly understood and implies that cells are maintained under physiological conditions and in a suitable aqueous medium that are capable of supporting the cells' viability and/or growth. For example, suitable media for supporting T-, B- and NK-cells viability and growth are optionally supplemented RPMI, DMEM and IMEM. For example, suitable media for supporting T cell viability and growth are Gibco CTS products and RPMI (with/without supplements such as cytokines, serum, etc.).

It was surprisingly found that nanoporous substrates featuring a defined pore diameter boost lymphocyte activation, expansion and for B-cells also antibody production. It was shown that the formation of actin-rich protrusions into specific pore diameter engineered nanoporous membranes significantly alters early cell signaling and gene expression of T-, B- and NK-cells.

The terms pore size and pore diameter, as used herein, generally indicate the distance between two opposite walls of a pore, i.e. the diameter of cylindrical pores or width of slip-shaped pores, and said distances are measured by their length in nm.

For example, the formation of dynamic protrusions leads to a spatiotemporal segregation of T-cell receptors excluding CD45 phosphatases from the nanopore protrusions, allowing sustained and augmented signaling in the assembled nanoclusters within the hotspots. The presence of a nanoporous surface prompts massive alterations in the transcriptional program and subsequently initiates activation of the cells even in the absence of stimulating biochemical signals. The synergistic combination of biomechanical and biochemical signals on porous surfaces presents a straightforward and low-cost strategy to boost T-cell activation and expansion.

Similar to the findings on T-cells, it was demonstrated that B-cells can also form actin-rich protrusions in pores of defined diameters. Additionally, the surface topography on which B cells are cultured can strongly enhance their activation (as demonstrated by measuring CD69 expression), proliferation (as demonstrated by measuring CTV dilution) and most importantly antibody production as measured by intracellular staining of IgG. The activation of B-cells is similar to that of T-cells and can be correlated with porous substrates directly regulating the gene expression program responsible for activation, proliferation and differentiation into a sub-species of B-cells with enhanced antibody production qualities.

The nanoporous substrate for practicing the method of the present invention exerts its dimensional constraints on T-, B- and NK-cells by pore diameter and its chemical nature or composition must merely be physiological, i.e. non-toxic, to improve T-, B- and NK-cell activation. Optionally, the nanoporous substrate is based on, optionally consists of a material selected from the group consisting of polymers, optionally polystyrene, polycarbonate, polydimethyl sulfate (PDMS), aluminum oxide, anodic aluminum oxide (AAO), titanium oxide, anodic titanium oxide (ATO), nanotubes, silicon, silicon oxide, silicon nitride, silicon carbide, diamond, diamond-like carbon, glassy carbon, optionally selected from the group consisting of polydimethyl sulfate (PDMS), silicon oxide, and AAO, optionally AAO.

The above nanoporous materials are state of the art and, for example, described in Ishibashi et al. 2009, Biomedical Microdevices 11 (2): 413-19; Malec et al. 2016, International Journal of Nanomedicine 11 (October): 5349-60; Cao et al. 2019, PNAS 116 (16): 7899-7904; and Oh et al. 2009, PNAS 106 (7): 2130-35).

The nanoporous substrate for practicing the method of the present invention may be uncoated because the physical dimensional constraints on T-, B- and NK-cells are sufficient for improving activation. However, the surface of the nanoporous substrate may be optionally coated with one or more compounds or compositions of choice, e.g. for improving surface properties of the substrate and/or for improving, e.g., differentiation, activation and/or growth of the cells.

For example, the nanoporous substrate may optionally be coated for T-cells with (poly)peptides, optionally antibodies, antibody fragments, antibody derivatives or antibody analogues, optionally against CD3 and/or CD28 and/or CD19, and/or with a polymer selected from the group consisting of poly-D-Lysine (PDL), poly-L-Lysine (PLL), and polyethylene glycol (PEG).

For example, the nanoporous substrate may optionally be coated for B-cells with (poly)peptides, optionally antibodies, antibody fragments, antibody derivatives or antibody analogues, optionally against CD40 and/or IgM while optionally supplemented with IL4 and/or CpG, and/or with a polymer selected from the group consisting of poly-D-Lysine (PDL), poly-L-Lysine (PLL), and polyethylene glycol (PEG).

For example, the nanoporous substrate may optionally be coated for NK-cells with (poly)peptides, optionally antibodies, antibody fragments, antibody derivatives or antibody analogues, optionally against CD16, NKG2D, SLAM family members and/or at least one of the natural cytotoxicity receptors NKp30, NKp44 and NKp46, and/or with a polymer selected from the group consisting of poly-D-Lysine (PDL), poly-L-Lysine (PLL), and polyethylene glycol (PEG).

In a specific embodiment for practicing the method of the present invention for activating T-cells, the nanoporous substrate is optionally coated with antibodies, antibody derivatives or antibody analogues against CD3 and/or CD28.

In a further specific embodiment for practicing the method of the present invention for activating B-cells, the nanoporous substrate is optionally coated with antibodies, antibody derivatives or antibody analogues against CD40 and/or IgM.

In a further specific embodiment for practicing the method of the present invention for activating NK-cells, the nanoporous substrate is optionally coated with antibodies, antibody derivatives or antibody analogues against CD16, NKG2D, SLAM family members and/or at least one of the natural cytotoxicity receptors NKp30, NKp44 and NKp46.

Antibodies, antibody derivatives such as functional fragments and functional derivatives thereof that specifically bind a polypeptide and are suitable for use in the method of the present invention are routinely available by hybridoma technology (Kohler and Milstein, Nature 256, 495-497, 1975), antibody phage display (Winter et al., Annu. Rev. Immunol. 12, 433-455, 1994), ribosome display (Schaffitzel et al., J. Immunol. Methods, 231, 119-135, 1999) and iterative colony filter screening (Giovannoni et al., Nucleic Acids Res. 29, E27, 2001) once the target antigen is available. Typical proteases for fragmenting antibodies into functional products are well-known. Other fragmentation techniques can be used as well as long as the resulting fragment has a specific high affinity and, preferably a dissociation constant in the micromolar to picomolar range. A very convenient antibody fragment for targeting and coating applications is the single-chain Fv fragment, in which a variable heavy and a variable light domain are joined together by a polypeptide linker. Other antibody fragments for coating the nanoporous substrates for practicing the present invention include Fab fragments, Fab 2 fragments, miniantibodies (also called small immune proteins), tandem scFv-scFv fusions as well as scFv fusions with suitable domains (e.g. with the Fc portion of an immunoglobulin). For a review on certain antibody formats, see Holliger P, Hudson P J.; Engineered antibody fragments and the rise of single domains. Nat Biotechnol. 2005 September, 23(9):1126-36). The term “functional derivative” of an antibody for use in the present invention is meant to include any antibody or fragment thereof that has been chemically or genetically modified in its amino acid sequence, e.g. by addition, substitution and/or deletion of amino acid residue(s) and/or has been chemically modified in at least one of its atoms and/or functional chemical groups, e.g. by additions, deletions, rearrangement, oxidation, reduction, etc. as long as the derivative has substantially the same binding affinity as to its original antigen and, preferably, has a dissociation constant in the micro-, nano- or picomolar range. Optionally, the antibody, fragment or functional derivative thereof for use in the invention is one that is selected from the group consisting of polyclonal antibodies, monoclonal antibodies, chimeric antibodies, humanized antibodies, CDR-grafted antibodies, Fv-fragments, Fab-fragments and Fab₂-fragments and antibody-like binding proteins, e.g. affilines, anticalines and aptamers. For a review of antibody-like binding proteins, i.e. antibody analogues, see Binz et al. on engineering binding proteins from non-immunoglobulin domains in Nature Biotechnology, Vol. 23, No. 10, October 2005, 12571268. The term “aptamer” describes nucleic acids that bind to a polypeptide with high affinity. Aptamers can be isolated from a large pool of different single-stranded RNA molecules by selection methods such as SELEX (see, e.g., Jayasena, Clin. Chem., 45, p. 1628-1650, (1999); Klug and Famulok, M. Mol. Biol. Rep., 20, p. 97-107 (1994); U.S. Pat. No. 5,582,981). Aptamers can also be synthesized and selected in their mirror form, for example, as the L-ribonucleotide (Nolte et al., Nat. Biotechnol., 14, pp. 1116-1119, (1996); Klussmann et al., Nat. Biotechnol., 14, p. 1112-1115, (1996)). Forms isolated in this way have the advantage that they are not degraded by naturally occurring ribonucleases and, therefore, have a greater stability. Another antibody-like binding protein and alternative to classical antibodies are the so-called “protein scaffolds”, for example, anticalines, that are based on lipocaline (Beste et al., Proc. Natl. Acad. Sci. USA, 96, p. 1898-1903, (1999)). The natural ligand binding sites of lipocalines, for example, of the retinol-binding protein or bilin-binding protein, can be changed, for example, by employing a “combinatorial protein design” approach, and in such a way that they bind selected haptens (Skerra, Biochem. Biophys. Acta, 1482, pp. 337-350, (2000)). For other protein scaffolds it is also known that they are alternatives for antibodies (Skerra, J. Mol. Recognit, 13, pp. 167-287, (2000)). (Hey, Trends in Biotechnology, 23, pp. 514-522, (2005)). In summary, the term functional antibody derivative is meant to include the above protein-derived alternatives for antibodies, i.e. antibody-like binding proteins, e.g. affilines, anticalines and aptamers, that specifically recognize a polypeptide, fragment or derivative thereof.

In a further embodiment, the method of the present invention is for producing activated T-cells, comprising the steps of
(i) providing and optionally surface cleaning the nanoporous substrate, optionally chemically and/or by plasma;
(ii) optionally surface functionalization, optionally with antibodies, (poly)peptides and/or polymers;
(iii) culturing T-cells on the surface of the porous substrate, optionally prior, during and/or after the activation; and
(iv) optionally co-culturing the T-cells with further different cell types.

In a further embodiment, the method of the present invention is for producing activated B-cells and/or for producing antibodies, comprising the steps of
(i) providing and optionally surface cleaning the nanoporous substrate, optionally chemically and/or by plasma;
(ii) optionally surface functionalization, optionally with antibodies, (poly)peptides and/or polymers;
(iii) culturing B-cells on the surface of the porous substrate, optionally prior, during and/or after the activation; and
(iv) optionally co-culturing the B-cells with further different cell types, optionally forming hybridoma cells.

In a further embodiment, the method of the present invention is for producing activated NK-cells, comprising the steps of
(i) providing and optionally surface cleaning the nanoporous substrate, optionally chemically and/or by plasma;
(ii) optionally surface functionalization, optionally with antibodies, (poly)peptides and/or polymers;
(iii) culturing NK-cells on the surface of the porous substrate, optionally prior, during and/or after the activation; and
(iv) optionally co-culturing the NK-cells with further different cell types.

In the following the present invention will be illustrated by means of specific experimental embodiments, none of which are to be interpreted as limiting the scope of the invention beyond the appended claims.

### FIGURES

Figures for T-Cells

FIG. 1 a is a confocal fluorescent microscopy image of Jurkat T-cells on a porous AAO with 200 nm pore diameter (3D view), scale bar 10 μm. Bottom—shows a schematic presentation of the actin-rich protrusions in T-cells (not to scale).

FIG. 1b shows the confocal fluorescent microscopy images of TCR from T-cells on a porous surface at the basal membrane (upper panel) or inside the pores (lower panel).

FIG. 1 c are bar diagrams showing activation of human primary T-cells on porous and non-porous surfaces. +/− signs indicate the presence of activation antibodies (αCD3/CD28) on the surface. CD69 expression and IL-2 secretion were measured after 24 hours, and CD25 expression was measured after 4 days. Three independent experiments were performed in duplicates or triplicates. The p-values were determined by two-sided Mann-Whitney tests in R.

FIG. 2 a) are two photographs showing the proliferation of primary human T-cells activated on different surfaces with aCD3/CD28 immobilized on the surface. The representative microscope images show proliferation of T-cells 5 days after activation (left: activated on non-porous surface, and right: activated on porous surface). The dark areas are clusters of proliferated T-cells. T-cells were activated on the non-porous or porous surfaces and were transferred to a plastic culture dish on day 3.

FIG. 2 b) is a bar plot of the statistical analysis of fold expansion of T-cells that were activated on porous or non-porous surfaces (2 independent experiments with 3 or 5 replicates). The cells were cultured for expansion according to the standard protocol. A 100-fold expansion was obtained when cells were activated on the porous surfaces.

Figures for B Cells

FIG. 3 is a column graph demonstrating early activation as checked via CD69 surface expression and flow cytometry. The further three column graphs relate to activated B-cells after 5 days that were stained intracellularly for IgG and analyzed by flow cytometry according to Example II.1 below. The graphs show percentages and representative histograms of proliferated (CTV-) cells, IgG+ cells among proliferated CTV cells as well as mean fluorescent intensity (MFI) of IgG staining of CTV-B cells on porous and non-porous surfaces, which represents the enhanced antibody production.

FIG. 4 a) is a column graph demonstrating early activation as checked via CD69 surface expression and flow cytometry. Primary B cell are solely activating via physical constraints of the nanopores for different nanopore dimensions. CD69 levels are shown for flat AAO and nanopores ranging from 20 nm, 100 nm, 200 nm till 400 nm diameter.

FIG. 4 b) is a column graph demonstrating early activation as verified via CD69 surface expression and flow cytometry. Primary B cell are activated using anti CD40 and anti IgM antibodies, immobilized on the AAO surface via Streptavidin, furthermore IL2 and IL4 are supplemented.

FIG. 4 c) (left) is a confocal fluorescent microscopy image of a primary B cell on a porous AAO with 200 nm pore diameter. Actin cytoskeleton is stained post-fixation using phalloidin-Alexa647. The red dots represent protrusions reaching into the AAO substrate. The image plane is 1 micrometer below AAO surface. (right) are normalized fluorescence intensity versus nanopore depth plots of confocal microscopy data for at least 50 protrusions of 5 B cells. Primary B cells 1 hour post seeding on 200 nm AAO surfaces with antibodies against IgM and CD40 are stained for pSyk, BCR and actin. Data shows enrichment of pSyk, BCR and actin within the protrusions.

### EXAMPLES

Materials & Methods

Porous anodic aluminum oxide (AAO) samples were round 13 mm diameter Whatman Anodisc Circles (Sigma-Aldrich), with 20 nm (WHA68097003), 100 nm (WHA68097013) and 200 nm (WHA68097023) pore diameter, or polystyrene/polycarbonate membranes (Isopore Membrane Filters, GTTP01300, HTTP01300) with 200 and 400 nm pore diameter.

High throughput RNA-sequencing, RNA extraction, differential expression analysis, plots (2D multidimensional scaling plot of filtered data with edgeR, volcano plots generated with EnhancedVolcano package (Blighe et al. EnhancedVolcano, R package version 1.4.0. (2019), gene-set enrichment analysis, flow cytometry, ELISA, immunostaining, optical microscopy, electron microscopy and statistical methods (using R, ±s.d., two-sided Mann-Whitney tests) were employed and routinely adapted to the T and B cells and the task.

ERK (Extracellular signal-regulated kinase) inhibition was achieved by incubating cells with U0126 (Abcam, ab120241) at different doses (0, 0.1, 1 and 10 μM diluted in Anhydrous DMSO (Sigma-Aldrich). The cells were then incubated with the porous/non-porous surfaces for 0-60 min and 24 hours. The cell concentration was 5×10⁵ cells/mL.

For Polymer coating the AAO membrane was first spin coated with photoresist (PR) AZ1518 (MicroChemicals GmbH) at 1750 rpm for 1 min and baked at 100° C. for 3 min. Reactive-ion etching (RIE) (Oxford, Plasmalab 80) was used to create and control different depths of nanohole on composite AAO-PR substrate. AAO-PR substrates were etched by RIE for 10, 15, 20 and 30 min with O₂: 20 sccm and RF power of 100 watt, achieving the respective depths of 0.5, 1, 2 and 4 μm. The AAO-PR nanohole was sputter coated with a 5 nm layer of Pt for SEM (Zeiss ULTRA 55) analysis.

### Example I T Cell Activation and Expansion

### Example I.1—Preparation of Non-Porous Aluminum Oxide Samples

Non-porous aluminum oxide samples were prepared by deposition of 40 nm of Al₂O₃ on a 13 mm round coverslip (1.5 H, 0117530, Marienfeld) using atomic layer deposition (ALD, Picosun Sunale R-150B). Briefly, ultrahigh-purity nitrogen carrier gas was purged at a flow rate of 200 sccm and a pressure of about 1 Torr was maintained. Al₂O₃ ALD was conducted with alternating exposures to trimethylaluminum (TMA) and water. TMA exposure and purge times were 0.1 and 4 s, respectively. The deposition was conducted at 150° C. with 400 cycles (0.1 nm/cycle).

### Example I.2 Cleaning the Surfaces

The samples were cleaned in an air-plasma (3 min at 18 W, using a PDC-32G; Harrick Plasma, USA). For glass coverslips, the samples were sonicated in acetone and isopropanol solutions (3 min each), rinsed with MilliQ water and dried with a nitrogen flow prior to the plasma cleaning. All samples were autoclaved at 120° C. for 2 h for experiments with primary human T-cells.

### Example I.3 Antibody Coating of the Surfaces

To coat the surface of the samples with activating antibodies (aCD3 and aCD28), a streptavidin intermediate was used. Streptavidin (Thermo Fisher Scientific, 434301) was diluted in PBS (ROTI Cell PBS, Carl Roth) to the concentration of 10 μg/mL. 100 μL of diluted streptavidin was added directly on top of the samples and was incubated for 30 min at room temperature. The samples were washed with 100 μL PBS (three times) before adding the activating antibodies.

Monoclonal CD3 and CD28 antibodies were used as activating antibodies. 100 μl of 5 μg/mL biotinylated CD3 (Thermo Fisher Scientific, 13-0037-82) and CD28 (Thermo Fisher Scientific, 13-0289-82) antibodies were added on top of each sample, and were incubated for 20 min at room temperature. The samples were then washed with PBS three times and were transferred to 24-well plates for cell seeding.

### Example 1.4 Cell Seeding

T-cells were freshly plated at a density of 2.5×10⁵ cells per well in a 24-well plate containing the 13 mm samples at the bottom. The medium consisted of RPMI (RPMI 1640, Invitrogen) with 10% fetal bovine serum (ATCC-LGC Standards). Cells were incubated for the planned duration at 37° C. and 5% CO 2, without any further supplements.

### Example 1.5 Human Primary T-Cells

Collection of plasma and PBMCs was approved by the Kantonale Ethikkommission Zurich (KEK-ZH-Nr. 2012-0111), and written consent was obtained from all subjects. A total of 6 separate healthy donors participated in this study. The average age of healthy donors was 35.7±3.1 (mean±s.d.). All experiments were performed in accordance with relevant guidelines and regulations.

### Example I.6 Isolation of the Primary Human T-Cells

Pan T-cells were purified from total peripheral blood from healthy adult volunteers. Freshly donated whole blood was first diluted to half with PBS (ROTI Cell PBS, Carl Roth) at room temperature. Then 35 mL of the diluted blood was added to 50 mL centrifuge tubes (SepMate, Stemcell Technologies) which were prefilled with 15 mL of density gradient medium (Lymphoprep, Stemcell Technologies). The samples were centrifuged at 1200×g for 10 min at room temperature. PBMCs in the top layer were washed with PBS and T-cells were isolated by negative selection using EasySep Human Naïve Pan T Cell Isolation Kit (Stemcell Technologies #17961), according to manufacturer's protocol. Briefly, 50 μL of isolation cocktail antibodies and 50 μL of TCR gamma/delta depletion cocktail were added to 1 mL of Lymphoprep-purified PBMCs for 5 min at room temperature. Next, 60 μL of RapidSpheres™ were added per 1 mL of sample for another 3 min at room temperature, the sample was topped up to 2.5 mL and placed on a magnet (EasySep magnet, StemCell Technologies) for 3 min. The unlabeled cells were poured into a new tube, washed, counted and used for further applications.

### Example I.7 T Cell Culture

Primary T cells were cultured in RPMI 1640 supplemented with 10% fetal bovine serum (FBS) (ATCC-LGC Standards), 1× penicillin/streptomycin, 2-ME (50 mM), nonessential amino acids (ThermoFischer Scientific), sodium pyruvate (ThermoFischer Scientific), HEPES (ThermoFischer Scientific) and glutamate (ThermoFischer Scientific). Jurkat cells (an immortalized line of human T-cell), were cultured in RPMI 1640 (Invitrogen) supplemented with 10% FBS and 1× penicillin-streptomycin (Invitrogen), and were kept in an incubator at 37° C. and 5% CO 2 for at least 3 days before measurements. To maintain the concentration of less than 10⁶ cells/mL, cells were split 1:4 with fresh media every 3-4 days. Prior to the experiments, cells were counted, centrifuged at 300×g for 3 minutes at room temperature and then resuspended in the fresh medium to a concentration of 5×10⁵ cells/mL. The amount of 0.5 mL of the cell suspension was then seeded into 24-well plates which contained the 13 mm samples in the bottom. The cells were incubated at 37° C. and 5% CO₂ before further analysis.

### Example I.8 Experimental Results and Conclusions

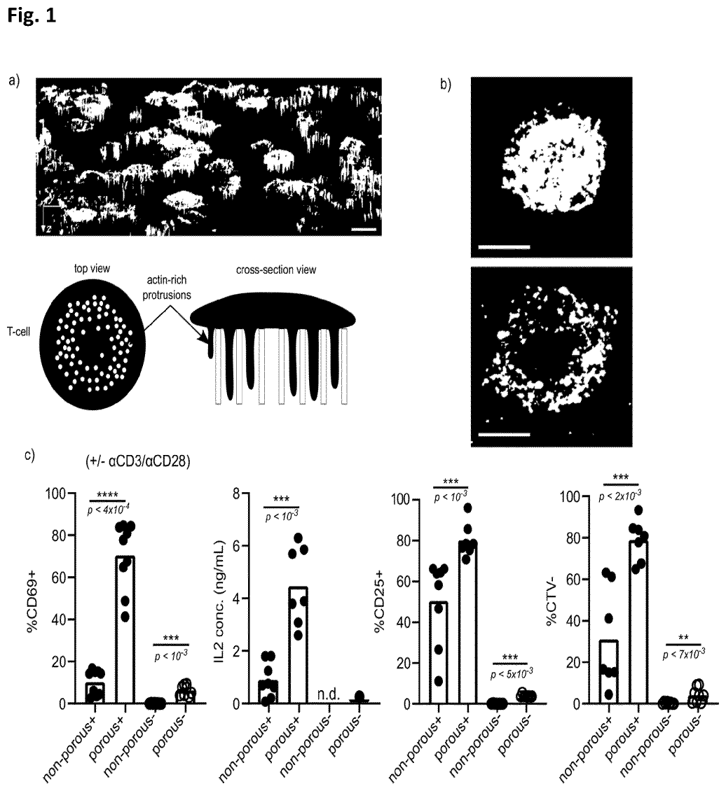

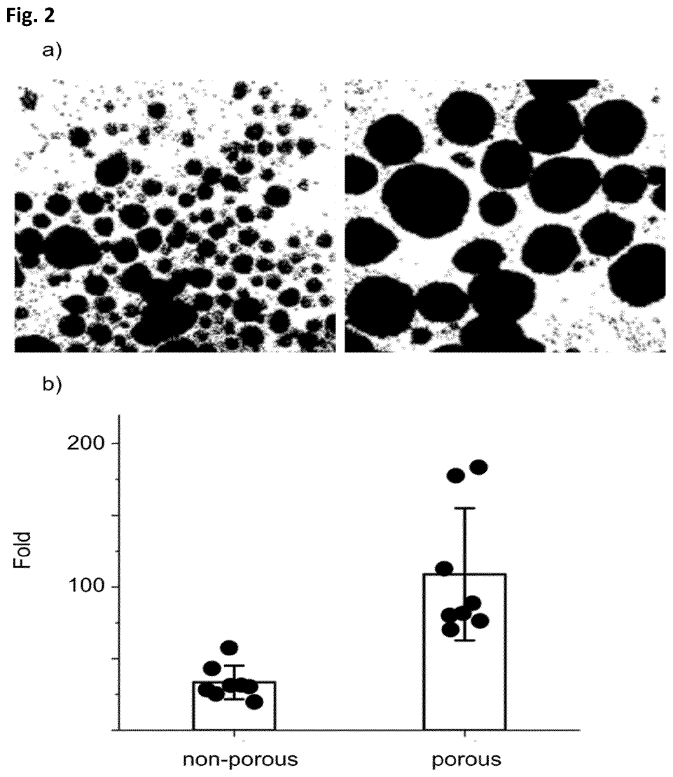

In summary, culturing T-cells on membranes with pores of about 100 to 500, in particular about 150 to 250 nm, for example about 200 nm, significantly boosts their activation and proliferation (see FIG. 2). Engineered nanoporous AAO membranes provide a guide for actin-rich nanoscale protrusions (see FIG. 1A), boosting T-cell activation and proliferation (see FIG. 1 C). The boosted activation of T-cells is facilitated via pore diameter-dependent segregation of membrane proteins, amplifying and sustaining the necessary signaling events. Kinetic segregation can be locally induced when T-cells protrude into nanoporous openings (see FIG. 1 B). Notably, cell signaling cascades and gene regulation are significantly altered in cells cultured on nanoporous membranes. ERK phosphorylation—a major component of MAP Kinase pathway and T-cell activation—is augmented and sustained in T-cells on nanoporous substrates, e.g. AAO, contributing to the activation of the cells. The synergistic combination of protrusion formation and antigen stimulation presents a simple and inexpensive strategy to optimize in vitro T-cell activation and can be used for adoptive T-cell therapy.

### Example II—B Cell Activation and Expansion on Porous Surface

### Example II.1 Surface Modification without IgM (Standard for B-Cell Activation)

Porous AAO with 200 nm pore diameter and non-porous glass surfaces were treated with oxygen plasma for 1 min, then coated with 10 ug/ml streptavidin containing PBS and subsequent incubation with 50 ug/ml ICAM for 30 min followed by biotinylated anti-CD40 for 15 min.

### Example II.2 Surface Modification with IgM

Additionally to the above described surface modification the role of surface presentation of anti-IgM aside anti-CD40 was investigated. Therefore, the surface with anti-IgM and a-CD40 for 15 min was co-incubated prior to cell seeding.

### Example II.3 B-Cell Seeding

Primary B-cells were purified from Peripheral Blood Mononuclear Cells (PBMCs) with an EasySep StemCell isolation kit, before seeding onto the modified AAO surface. B-cells were cultured for different times in the presence of 30 U/ml IL2 and 20 ng/ml IL4 (to compare porous with non-porous substrates.

### Example II.4 B-Cell Activation Via aCD40

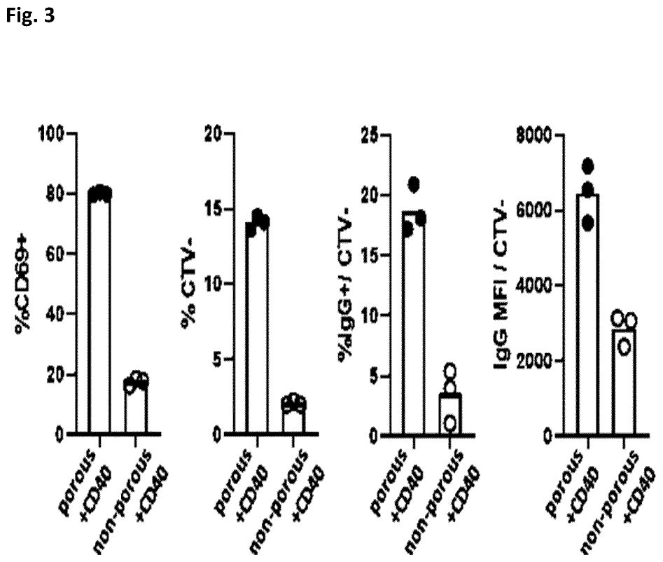

After 30 min B-Cell seeding, samples were fixed with PFA, stained with phalloidin-Alexa647 and analysed by confocal microscopy. Early activation was checked via CD69 surface expression and flow cytometry (FIG. 3). After 5 days, activated B cells were stained intracellularly for IgG and analysed by flow cytometry. Shown are percentages and representative histograms of proliferated (CTV-) cells, IgG+ cells among proliferated CTV-cells as well as mean fluorescent intensity (MFI) of IgG staining of CTV-B cells on porous and non-porous surfaces, which represents the enhanced antibody production (FIG. 3).

### Example II.5 B-Cell Activation Via Anti-CD40 and Anti-IgM

Porous AAO with 200 nm pore diameter and non-porous glass surfaces were treated with oxygen plasma for 1 min, then coated with 10 ug/ml streptavidin for 30 min followed by biotinylated anti-CD40 and anti-IgM for 15 min. Primary B cells were purified from PBMCs with an EasySep StemCell isolation kit, labeled with CTV for 10 min at 37° C. and cultured on the coated surfaces in the presence of 30 U/ml IL2 and 20 ng/ml IL4. After 24 h, activated B cells were stained for CD69 surface expression and measured by flow cytometry.

### Example II.6 Experimental Results and Conclusions

Similar to the above-described results for T cells it was demonstrated that B cells can form actin-rich protrusions in pores of about 100 to 500, in particular about 150 to 250 nm, for example about 200 nm. It was found that the surface topography on which B cells are cultured strongly enhances their activation (as measured and shown by CD69 expression), proliferation (as measured by CTV dilution) and most importantly antibody production (as measured by intracellular staining of IgG) (see FIG. 3). The activation for B-cells is similar to that of T-cells and is correlated with porous substrates directly regulating the gene expression program responsible for activation, proliferation and differentiation into a sub-species of B-cells with enhanced antibody production qualities.

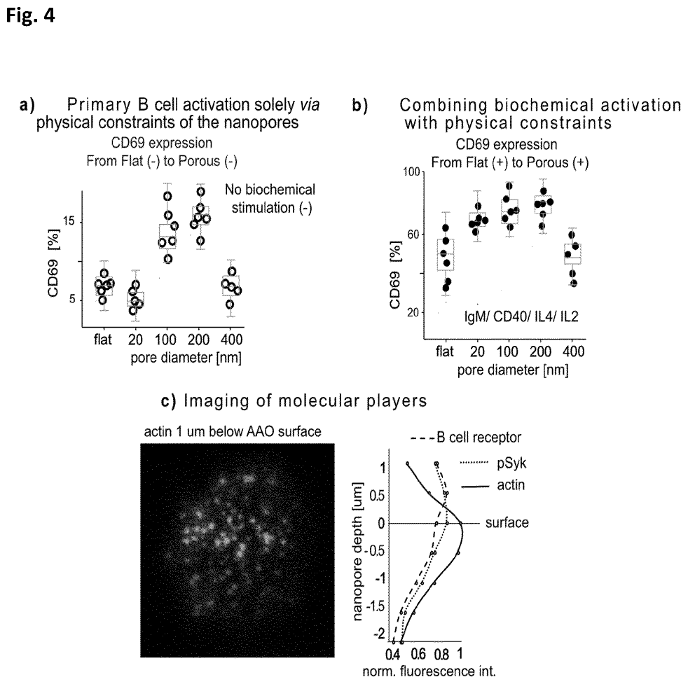

In summary, culturing B-cells on membranes with pores of about 100 to 500, in particular about 150 to 250 nm, significantly boosts their activation, proliferation and antibody production (see FIG. 3). Engineered nanoporous AAO membranes provides a guide for actin-rich nanoscale protrusions (see FIG. 3), boosting B-cell activation and proliferation (see FIG. 3). The boosted activation of B-cells is facilitated via pore diameter-dependent segregation of membrane proteins, amplifying and sustaining the necessary signaling events (see FIG. 4). Kinetic segregation is locally induced when B-cells protrude into nanoporous openings. The synergistic combination of protrusion formation and antigen stimulation presents a simple and inexpensive strategy to optimize in vitro B-cell activation and is of value for protocols for adoptive cell therapy and antibody production.

Most current protocols for activating naive or memory B cells involve costimulation of CD40 and IL4 with or without the presence of soluble TLR ligands such as CpG. Similar to the findings on T cells, it was demonstrated that B cells can form actin-rich protrusions in specifically diameter-limited pores. Additionally, the surface topography on which B cells are cultured can strongly enhance their activation (as measured by CD69 expression), proliferation (measured by CTV dilution) and most importantly antibody production as measured by intracellular staining of IgG. The activation for B-cells is similar to that of T-cells and can be correlated with porous substrates directly regulating the gene expression program responsible for activation, proliferation and differentiation into a sub-species of B-cells with enhanced antibody production quality.

### Example III NK-Cell Activation and Expansion

### Example III.1 Surface Modification with aCD16 (Standard for NK-Cell Activation)

Porous AAO with about 200 nm pore diameter and non-porous glass surfaces can be treated with oxygen plasma for 1 min, then coated with 10 ug/ml streptavidin containing PBS and subsequently incubated with biotinylated anti-CD16 to activate NK cells.

### Example III.2 Surface Modification with Antibodies Against NKG2D, SLAM Family Members and the Natural Cytotoxicity Receptors NKp30, NKp44 and NKp46

Additionally to the above described surface modification the role of surface presentation of antibodies against NKG2D, SLAM family members and the natural cytotoxicity receptors NKp30, NKp44 and NKp46 can be optionally used for NK cell activation. Therefore, the nanoporous surface can optionally be co-incubated with one or more of the mentioned antibodies, optionally against CD16 prior to cell seeding.

### Example III.3 NK-Cell Seeding

Primary NK-cells can be purified from Peripheral Blood Mononuclear Cells (PBMCs) with an EasySep StemCell isolation kit, before seeding onto the modified AAO surface. NK-cells can be cultured for different times in the presence of TGF-β and/or IL15 and/or IL18 (to compare porous with non-porous substrates).

1. A method for the cultivation of lymphocytes (T-, B- and NK-cells) comprising the step of culturing the cells in a suitable cell growth medium on a nanoporous substrate, characterized in that the nanoporous substrate has a pore diameter in the range of about 100 to 500 nm.

2. The method according to claim 1, wherein the nanoporous substrate has a pore diameter in the range of 150 to 400 or 150 to 250, optionally of about 200 nm.

3. The method according to claim 1 or 2, wherein the nanoporous substrate is based on, optionally consists of a material selected from the group consisting of polymers, optionally polystyrene, polycarbonate, polydimethyl sulfate (PDMS), aluminum oxide, anodic aluminum oxide (AAO), titanium oxide, anodic titanium oxide (ATO), nanotubes, silicon, silicon oxide, silicon nitride, silicon carbide, diamond, diamond-like carbon, glassy carbon, optionally selected from the group consisting of polydimethyl sulfate (PDMS), silicon oxide, and AAO, optionally AAO.

4. The method according to any of claims 1 to 3, wherein the nanoporous substrate is coated,
(i) optionally coated for T-cells with (poly)peptides, optionally antibodies, antibody fragments, antibody derivatives or antibody analogues, optionally against CD3 and/or CD28 and/or CD19, and/or with a polymer selected from the group consisting of poly-D-Lysine (PDL), poly-L-Lysine (PLL), and polyethylene glycol (PEG);
(ii) optionally coated for B-cells with (poly)peptides, optionally antibodies, antibody fragments, antibody derivatives or antibody analogues, optionally against CD40 and/or IgM while optionally supplemented with IL4 and/or CpG, and/or with a polymer selected from the group consisting of poly-D-Lysine (PDL), poly-L-Lysine (PLL), and polyethylene glycol (PEG); and
(iii) optionally coated for NK-cells with (poly)peptides, optionally antibodies, antibody fragments, antibody derivatives or antibody analogues, optionally against CD16, NKG2D, SLAM family members and/or at least one of the natural cytotoxicity receptors NKp30, NKp44 and NKp46, and/or with a polymer selected from the group consisting of poly-D-Lysine (PDL), poly-L-Lysine (PLL), and polyethylene glycol (PEG).

5. The method according to any of claims 1 to 4 for activating T-cells, wherein the nanoporous substrate is coated with antibodies, antibody fragments, antibody derivatives or antibody analogues against CD3 and/or CD28.

6. The method according to any of claims 1 to 4 for activating B-cells, wherein the nanoporous substrate is coated with antibodies, antibody fragments, antibody derivatives or antibody analogues against aCD40 and/or IgM.

7. The method according to any of claims 1 to 4 for activating NK-cells, wherein the nanoporous substrate is coated with antibodies, antibody fragments, antibody derivatives or antibody analogues against CD16, NKG2D, SLAM family members and/or at least one of the natural cytotoxicity receptors NKp30, NKp44 and NKp46.

8. The method according to any of claims 1 to 4 and 5 for producing activated T-cells, comprising the steps of
(i) providing and optionally surface cleaning the nanoporous substrate, optionally chemically and/or by plasma;
(ii) optionally surface functionalization, optionally with antibodies, (poly)peptides and/or polymers;
(iii) culturing T-cells, optionally genetically engineered T-cells, optionally CAR T-cells, on the surface of the porous substrate, optionally prior, during and/or after the activation; and
(iv) optionally co-culturing the T-cells with further different cell types.

9. The method according to any of claims 1 to 4 and 6 for producing activated B-cells and/or for producing antibodies, comprising the steps of
(i) providing and optionally surface cleaning the nanoporous substrate, optionally chemically and/or by plasma;
(ii) optionally surface functionalization, optionally with antibodies, (poly)peptides and/or polymers;
(v) culturing B-cells, optionally genetically engineered B-cells, on the surface of the porous substrate, optionally prior, during and/or after the activation; and
(vi) optionally co-culturing the B-cells with further different cell types, optionally forming hybridoma cells.

10. The method according to any of claims 1 to 4 and 7 for producing activated NK-cells, comprising the steps of
(i) providing and optionally surface cleaning the nanoporous substrate, optionally chemically and/or by plasma;
(ii) optionally surface functionalization, optionally with antibodies, (poly)peptides and/or polymers;
(vii) culturing NK-cells, optionally genetically engineered NK-cells, optionally CAR NK Cells on the surface of the porous substrate, optionally prior, during and/or after the activation; and
(viii) optionally co-culturing the NK-cells with further different cell types.
